(19) United States
(12) Patent Application Publication
(10) Pub. No.: US 2018/0292390 A1
(43) Pub. Date: Oct. 11, 2018

(54) NOVEL SLICE CULTURES AND METHODS FOR DIAGNOSING NEURONAL DEGENERATION DISEASES

(71) Applicant: ROWAN UNIVERSITY, Glassboro, NJ (US)

(72) Inventors: Venkat Venkataraman, Glassboro, NJ (US); Robert G. Nagele, Turnersville, NJ (US); Hao Wu, Glassboro, NJ (US)

(73) Assignee: Rowan University, Glassboro, NJ (US)

(21) Appl. No.: 15/567,897

(22) PCT Filed: Apr. 25, 2016
(86) PCT No.: PCT/US16/29182

§ 371 Date: Oct. 19, 2017

Related U.S. Application Data
(60) Provisional application No. 62/151,698, filed on Apr. 23, 2015.

(51) Int. Cl. G01N 33/50

(52) U.S. Cl. CPC G01N 33/5082; G01N 33/5091; G01N 2800/28; G01N 2800/2835; G01N 2800/2857; G01N 2800/2821

(57) ABSTRACT

The present invention relates novel slice culture systems which provide a quick, simple, and effective tool for investigating pathological changes associated with AD. Also provided are methods of diagnosing AD and identifying potentially therapeutic compounds.

### CROSS-REFERENCE TO RELATED APPLICATIONS

The present application claims priority under 35 U.S.C. § 119(e) of U.S. Provisional Application No. 62/151,698, filed Apr. 23, 2015, the disclosure of which is hereby incorporated by reference in its entirety.

### FIELD OF THE INVENTION

The present invention relates to slice cultures for studying various neuronal degeneration diseases and for developing novel approaches to diagnosing and treating such diseases.

### BACKGROUND

Alzheimer's disease (AD) is a progressive neurological disease, mainly of the elderly, that is hallmarked by cognitive decline which results in loss of language and communication skills, difficulty in learning, loss of memory, and alterations in personality and mood. AD is the most common form of dementia, currently affecting over 5.5 million people in the United States and more than 35 million people worldwide. The pathological changes seen in AD include synaptic loss, dendrite retraction, neuronal cell death, inflammation, astrocyte activation, cerebral amyloid angiopathy, blood-brain barrier (BBB) breakdown, and the accumulation of amyloid peptide 1-42 (Aβ42) within neurons and plaques throughout the hippocampus and cerebral cortex (Clifford, et al., Brain Res. 1142, 223-36, 2007; Clifford, et al., Brain Res. 1234, 158-71, 2008; Rogers, et al., Biochem Soc Trans. 36, 1282-7, 2008; Selkoe, et al., Nutr Rev. 65, S239-43, 2007.). Breakdown of the BBB is a particularly important development in AD progression, as it allows for the leakage of potentially damaging humoral elements into the brain parenchyma. The BBB is comprised of specialized vascular endothelial cells that are connected to one another via a variety of tight junction proteins. The endothelial cells of the BBB differ from those in other parts of the mammalian body in that they lack fenestrations and therefore do not allow for free exchange of solutes between the blood and the brain parenchyma. Additionally, astrocytic foot processes wrap around the blood vessels and play an important role in allowing endothelial cells of the BBB to form their normally protective, tight seal. Injury or disease of the CNS, such as AD, causes gliosis, resulting in activation of astrocytes and an increased expression of glial fibrillary acidic protein (GFAP) in these cells. When BBB breech occurs in the AD brain, it allows for the extravasation of blood-borne Aβ42, brain-reactive autoantibodies, and inflammatory cells of the immune system into the normally immune-privileged brain parenchyma (Grammas, J Neuroinflammation. 8, 26, 2011). Access of the previously excluded and potentially damaging blood-borne plasma elements to the brain interstitium, results in disruption of brain homeostasis, impaired neuronal function, and eventually, neuronal damage and loss. These deleterious effects on neurons are apparently buffered somewhat by activation of neuronal repair mechanisms, one of which involves neuronal expression of vimentin. Vimentin is an intermediate filament protein that is found primarily in endothelial cells and developing neurons. Vimentin expression in neurons has been linked temporally and spatially to dendrite repair in neurons of the cerebral cortex in AD brains and mouse brains subjected to traumatic injury (Levin et al., Brain Res. 1298, 194-207, 2009). Thus BBB breakdown is a key event in initiating damage and damage responses in neurons in AD.

Many inflammatory mediators and cytokines are thought to contribute to BBB breakdown, including bradykinin, nitric oxide, oxygen radicals, and histamine. Histamine is a proinflammatory mediator derived from the amino acid histidine. It is present throughout the mammalian body, predominantly localized to mast cell granules and basophils. Histamine also acts as a neurotransmitter, and is released by histaminergic neurons of the tuberomamillary nucleus of the posterior hypothalamus. Upon injury or trauma, an inflammatory response occurs that results in the release of histamine. Histamine causes an increase in BBB permeability by opening the inter-endothelial cell tight junctions (Sakurai, et al., Inflamm Res. 58 Suppl 1, 34-5, 2009). It exerts its effects on endothelial cells by engaging a series of secondary messenger pathways. Several in vivo studies have shown that histamine, whether applied luminally or abluminally to microvasculature of the brain, results in increased permeability of the BBB (Revest et al., Brain Res. 652, 76-82, 1994). Moreover, other studies have shown that histamine induces a swelling of perivascular glial foot processes when applied luminally via carotid artery infusion. While histamine has been previously shown to induce BBB permeability, it is not yet known if this leads to generation of additional brain pathologies, including those that are seen in AD.

The BBB is comprised of specialized vascular endothelial cells that are connected to one another via a variety of tight junction proteins. The endothelial cells of the BBB differ from those in other parts of the mammalian body in that they lack fenestrations and therefore do not allow for free exchange of solutes between the blood and the brain parenchyma. Additionally, astrocytic foot processes wrap around the blood vessels and play an important role in allowing endothelial cells of the BBB to form their normally protective, tight seal. Injury or disease of the CNS, such as AD, causes gliosis, resulting in activation of astrocytes and an increased expression of glial fibrillary acidic protein (GFAP) in these cells. When BBB breech occurs in the AD brain, it allows for the extravasation of blood-borne Aβ42, brain-reactive autoantibodies, and inflammatory cells of the immune system into the normally immune-privileged brain parenchyma. Access of the previously excluded and potentially damaging blood-borne plasma elements to the brain interstitium, results in disruption of brain homeostasis, impaired neuronal function, and eventually, neuronal damage and loss. These deleterious effects on neurons are apparently buffered somewhat by activation of neuronal repair mechanisms, one of which involves neuronal expression of vimentin. Vimentin is an intermediate filament protein that is found primarily in endothelial cells and developing neurons. Recently, vimentin expression in neurons has been linked temporally and spatially to dendrite repair in neurons of the cerebral cortex in AD brains and mouse brains subjected to traumatic injury. Thus BBB breakdown is a key event in initiating damage and damage responses in neurons in AD.

There exists a need for in vitro culture systems for studying AD as well as non-invasive diagnostic methods and treatment of such diseases.

### SUMMARY OF THE INVENTION

The present invention is designed to provide an in vitro slice culture system that is capable of mirroring the pathological changes in neuronal degeneration diseases such as AD. In one aspect of the invention, multiple identified biomarkers allow the study of the biological mechanism underlining the diseases and the development of novel treatment regimens. In another aspect of the invention, the presently disclosed slice culture system can be employed to identify new therapeutic compounds for neurological diseases, particularly related to breakage in blood brain barrier. In yet another aspect of the invention, the retinal slice culture system of the present invention provides novel non-invasive diagnostic approaches to neuronal degeneration diseases.

In another aspect of the present invention, mammalian brain slice cultures (MBO) treated with histamine provide a rapid model system for studying the effects of some cellular pathologies associated with AD and other neuro-inflammatory diseases. In yet another embodiment, such system may be employed to reverse or mitigate these pathological changes that occur in patients suffering from progressive neurological diseases.

In one aspect of the present invention, a novel slice culture system is provided, where a chemical or an agent induce pathological changes consistent with those of a neuronal degeneration diseases. In one embodiment, the system is prepared from an organotypic brain slice culture. In yet another embodiment, the system is prepared from a retinal slice culture.

In one embodiment, the slice culture system may be used in diagnosing or identifying the progression and extent of neurological diseases including Amyotrophic lateral sclerosis (ALS), Parkinson's disease (PD), Alzheimer's disease (AD), and Huntington's disease (HD). In some embodiments, the organotypic brain slice culture exhibits characteristics of AD such as leaky vessels, GFAP-positive astrocytes, and vimentin-positive neurons. In some embodiments, the retinal slice culture mimics the pathological changes in a patient retina including up-regulation of GFAP and down-regulation of Microtubule-associated protein 2 (MAP2). In some embodiments, the AD is Early-Stage AD.

In one aspect of the invention, chemical or agents that can be used to induce pathological changes in the slice culture include inflammation associated reagents such as histamine, TNF alpha, lipopolysaccharide, aluminum chloride, serotonin, purine nucleotides such as ATP, ADP, AMP, cytokines such as interleukin 1 α, growth factors such as monocyte chemoattractant protein (MCP-1), activators of the phosphatidylinositol/Akt pathway such as VEGF, oxidative stress associated reagents such as generators of free radicals and nitric oxide, or extracts from natural compounds such as turmeric or conditions of culture such as hypoxic or hyperbaric which individually or collectively in at least certain combinations, induce leaky vessels, GFAP-positive astrocytes, and vimentin-positive neurons in the slice culture of the present invention. In another embodiment, the chemical or agents of choice may be an analog or derivatives of histamine and can also be used for inducing desirable pathologies in the slice culture.

In one aspect of the invention, the slice culture is derived from human or other animals including rats, rabbits, guinea pigs and mice. In one embodiment, the animals may be of wide type of transgenic.

In another aspect, the present invention provides a method of preparing a slice culture system for studying neuronal degeneration diseases. In at least one embodiment, the method follow the steps of treating an organotypic brain slice culture or a retinal slice culture with an agent such as histamine and allowing sufficient exposure time to such agents so that the cells exhibit the same behavior as cell or in patients suffering from a neurological disease such as AD, PD, ALS, Huntington's disease or the like.

In another aspect, the present invention provides a method of evaluating the therapeutic effect of a compound comprising: contacting a test compound with a test slice culture, wherein said slice culture is treated with an agent before, after, or at the same time of contacting with the test compound, said agent induces one or more biomarkers of a neuronal degeneration disease, and said slice culture is selected from an organotypic brain slice culture and a retinal slice culture; measuring one or more biomarkers of a neuronal degeneration disease in said test slice culture; comparing the measurement of said biomarkers with a control or a baseline level to evaluate the therapeutic effect of the compound for treating or preventing the neuronal degeneration disease. The method of the present invention is applicable to evaluating compounds useful in managing various diseases including for example ALS, PD, AD and HD. The biomarkers may include Immunoglobulin G (IgG), cytoskeletal proteins such as GFAP, MAP2 and vimentin, calcium binding proteins such as S100B and visinin-like proteins, proteins that impact the cyclic GMP pathway such as membrane guanylate cyclases and their modulators In at least one embodiment, agents inhibiting or reducing the activity of the AD biomarkers may be used as therapeutic agents to treat AD, including Early-Stage AD.

In another aspect of the present invention, the system promotes modified cellular expression of certain cellular proteins such as Glial fibrillary acidic protein (GFAP), and vimentin in the organotypic brain slice culture and GFAP and Microtubule-associated protein 2 (MAP2) in the retinal slice culture. A more detailed explanation of the invention is provided below.

### BRIEF DESCRIPTION OF THE DRAWINGS

FIG. 1A and FIG. 1B illustrate the blood vessels in a diseased (AD) state and in a normal state, respectively.

FIG. 1C and FIG. 1D illustrate the astrocytes in a diseased (AD) state and in a normal state, respectively.

FIG. 1E and FIG. 1F illustrate the vimentin expression in a diseased (AD) state and in a normal state, respectively.

FIG. 2A1, 2A2 and 2A3 illustrate the blood vessels in untreated control MBOs.

FIG. 2B1, 2B2 and 2B3 illustrate the blood vessels in histamine-treated MBOs.

FIG. 3A1, 3A2 and 3A3 illustrate the GFAP cells in untreated control MBOs.

FIG. 3B1, 3B2 and 3B3 illustrate the GFAP cells in histamine-treated MBOs.

FIG. 4A1, 4A2 and 4A3 illustrate the vimentin expression in untreated control MBOs.

FIG. 4B1, 4B2 and 4B3 illustrate the vimentin expression in histamine-treated MBOs.

FIG. 5A illustrates the comparison of blood vessels in histamine-treated MBOs and untreated controls.

FIG. 5B illustrates the comparison of GFAP in histamine-treated MBOs and untreated controls.

FIG. 5C illustrates the comparison of Viementin in histamine-treated MBOs and untreated controls.

FIG. 6A illustrates the AD-like pathologies in 1 mm MBOs.

FIG. 6B illustrates the AD-like pathologies in 2 mm MBOs.

FIG. 7 illustrates a section of retina with BRB breakdown and extravasated IgG surrounding blood vessels in retinal slice cultures between histamine-treated model and untreated model.

FIG. 8 illustrates GFAP observed in retinal slice cultures after treated with histamine for different lengths of time.

FIG. 9 illustrates MAP2 observed in retinal slice cultures after treated with histamine for different lengths of time.

FIG. 10 illustrates the damage control response by LXA4 observed after simultaneous treatment of retinal slices with histamine.

FIG. 11 illustrates measurements of the different layers of retina and the effect of the loss of S100B function (S100Bko) as well as the blood retinal barrier breach.

FIG. 12 illustrates the specific changes in that of the inner and outer segment layers of the photoreceptors upon treatment with different “hits” that lead to Alzheimer's disease.

FIG. 13 illustrates specific changes observed in the cone types of photoreceptors measures by staining with cone arrestin.

FIG. 14A represents western blot analysis for whole brain protein extract from WT mice. FIG. 14B represents western blot analysis for swine retina protein extract.

FIG. 15 illustrates the width of the Müller cell processes from all groups. Statistical significance is determined by two-tailed Student's t test. *, P<0.05; **, P<0.01; ***, P<0.001. Mean±SEM is plotted in the graph.

FIG. 16 illustrates the density of continuous, MAP2-positive ganglion cell processes from all groups are presented. Statistical significance is determined by two-tailed Student's t test. *, P<0.05; **, P<0.01; ***, P<0.001. Mean±SEM is plotted in the graph.

FIG. 17 illustrates that sera from control, aged and AD patients recognize NCS proteins differentially.

FIG. 18 illustrates percentages of IgG-positive area (FIG. 18A and FIG. 18B), mean grey values (FIG. 18C) and IgG-positive neurons per square mm (FIG. 18D) from each group were measured by ImageJ and plotted in the graph. Student t-tests were performed between indicated groups. *, P<0.05; **, P<0.01; ***, P<0.001.

FIG. 19 illustrates the fluorescence intensity (quantified as mean grey values) as measured by ImageJ. Student t-tests were performed between indicated groups. *, P<0.05; **, P<0.01; ***, P<0.001.

FIG. 20 illustrates the fluorescence intensity (quantified as mean grey values) as measured by ImageJ. Student t-tests were performed between indicated groups. *, P<0.05; **, P<0.01; ***, P<0.001.

FIG. 21 illustrates the number of positive cells per unit area as determined through ImageJ. Student t-tests were performed between indicated groups. *, P<0.05; **, P<0.01; ***, P<0.001.

### DETAILED DESCRIPTION

The present invention is generally related to a slice culture system for studying neuronal degeneration diseases and evaluating or identifying therapeutic agents for treating or preventing such diseases. In one aspect of the invention, a slice culture system is defined that includes an organotypic tissue slice culture, preferably brain slice culture or a retinal slice culture, wherein after sufficient exposure to a chemical, agent or a composition such tissue exhibit the same characteristics as a tissue obtained from a patient that is suffering from a neuronal degeneration disease. In another aspect of the invention, the presently described system can be employed as an assay for determining potential drug candidates for each of the studied neuronal disease. In yet another aspect of the invention, a retinal slice culture of the present invention may be used for a method of diagnosing neuronal degeneration diseases in a person in need thereof.

Throughout this patent document, various publications are referenced. The disclosures of these publications in their entireties are hereby incorporated by reference into this application in order to more fully describe the state of the art to which the disclosed matter pertains. While the following text may reference or exemplify specific slice cultures, it is not intended to limit the scope of the invention to such particular reference or examples. Various modifications may be made by those skilled in the art, in view of practical and economic considerations, such as the biomarkers and the antibodies for detecting such biomarkers. In order to more clearly and concisely describe the subject matter of the claims, the following definitions are intended to provide guidance as to the meaning of terms used herein.

As used herein, the term “organotypic tissue slice culture” refers to suitable tissue slices that are removed from an organ and can be maintained in a suitable culture and medium to continue to develop as it would have in that same organ, but instead is maintained for further analysis or research. For example, slices of CNS tissue may be maintained in culture having basic requirements such as culture medium, sufficient oxygenation, and incubation at a suitable temperature so that never cells continue to differentiate and develop a tissue organization that closely resembles that observed in situ including preserving or developing their respective three dimensional structures. For example “brain slice culture” refers to sections or explants of brain tissue which are maintained in culture. Organotypic brain slice culture can employ sections of whole brain tissue or explants obtained from specific regions of the brain. Any region can be used to generate an organotypic brain slice culture, including for example the hippocampus or cortex region.

“About” means the referenced numeric indication plus or minus 5% of that referenced numeric indication.

The articles “a” and “an” as used herein mean “one or more” or “at least one,” unless otherwise indicated. That is, reference to any element of the present invention by the indefinite article “a” or “an” does not exclude the possibility that more than one of the element is present.

In one aspect of the invention, a system for studying neuronal degeneration diseases is provided. The system typically comprises an organotypic slice culture, preferably a brain slice culture or a retinal slice culture, treated with an agent that induces cellular conditions that mimic the pathological changes of patients suffering from certain neuronal degeneration diseases. In one embodiment, the agent may be applied to the slice culture directly, indirectly, during the incubation within an agent containing composition, or prior to the tissue being incubated in a composition or medium containing the inducing agent.

For example, in an organotypic brain slice culture of the present invention for studying Alzheimer's disease (AD), the brain slice culture is incubated in a medium containing histamine. Upon sufficient time of incubation, the tissue develops the leaky vessels which are respectively manifested by IgG leakage into brain tissue parenchyma. In addition, the astrocyte activation is evidenced by an increased expression of GFAP. The cellular neuronal damage-response is shown by vimentin expression in the cells.

In a retinal model of slice culture system of the present invention, the pathological changes provided may be represented by an up-regulation of GFAP and down-regulation of Microtubule-associated protein 2 (MAP2).

In at least one aspect of the present invention, the slice culture system may be used to study the extent, the stage and the progression of the particular disease such as ALS, PD, AD and HD. In at least one embodiment, a system is described for studying neuronal degeneration diseases, wherein a slice tissue culture is pretreated with at least one chemical or an agent capable of inducing a pathological modifications mimicking the same pathological conditions observed in tissues with the neuronal degeneration disease under investigation. In at least one embodiment, the preferred slice culture is selected from organotypic brain slice culture and retinal slice culture.

In yet another embodiment the system described herein are suitable for investigating the progression of the neuronal degeneration conditions such as ALS, PD, AD, and HD. In at least one embodiment, for example, the incubated organotypic brain slice culture may exhibit leaky vessels, GFAP-positive astrocytes, and vimentin-positive neurons. In another embodiment, the incubated retinal culture exhibits at least one characteristic such as up-regulation of GFAP or down-regulation of Microtubule-associated protein 2 (MAP2).

In yet another embodiment, the chemical or agents of choice is histamine, serotonin, inflammation associated reagents such as histamine, TNF alpha, lipopolysaccharide, aluminum chloride, serotonin,purine nucleotides such as ATP, ADP, AMP, cytokines such as interleukin 1a, growth factors such as monocyte chemoattractant protein (MCP-1), activators of the phosphatidylinositollAkt pathway such as VEGF, oxidative stress associated reagents such as generators of free radicals and nitric oxide, or extracts from natural compounds such as turmeric or conditions of cell culture such as hypoxic or hyperbaric. Various chemicals or agents that promote a cellular event may be applied, alone or in combination with other agent, to a slice culture as a pretreatment agent. One agent of particular interest is histamine, which has been found to elicit responses that included a breakdown of the BBB, astrocyte activation, and the initiation of a neuronal damage-response. Suitable agents for the present invention also include analogs and derivatives of histamine.

In a preferred embodiment, histamine is a chemical of choice to induce cellular pathologies consistent with those seen for example in AD, when administered in slice cultures such as mouse brain organotypic slice cultures (MBOs). As further illustrated in the figures and examples, histamine is a potent mediator of pathology, inducing BBB breakdown, gliosis with astrocyte activation, and the initiation of vimentin expression in neurons as part of a damage response mechanism.

Histamine-treated MBOs show multiple pathological changes relating to AD. First, an increased BBB permeability demonstrated by extravasation of serum components as indicated by IgG leakage has been observed. Second, significant inflammation as measured by increased gliosis, is a common pathology to AD. Third, Histamine treatment of MBOs also results in significant peri-nuclear vimentin expression within the cell bodies of neurons. Vimentin expression is mainly restricted to the vascular epithelium of control MBOs under normal conditions.

The slice tissue cells of the presently described system can be obtained from any mammal, such as rat, mice, chimpanzee, humans or other suitable animal model. In at least one embodiment, the tissue may also possess specific three dimensional features consistent with its source. In another embodiment, various cellular responses that correlate with symptoms of neuronal degeneration diseases can be identified in early stages of the disease of interest. For example, in an organotypic brain slice culture of the present invention, the leaky vessels are manifested by IgG leakage into brain parenchyma. The astrocyte activation is evidenced by an increased expression of GFAP and the cellular neuronal damage can be predicted by extent and degree of vimentin expression in the cells. Depending on how advanced a disease may be in a given patients, measurements directed to extent of IgG leakage, astrocyte activation or vimentin cellular expression can be employed individually or collectively as means to characterize the progression, the prognosis or even choice of treatment for the neurological diseases. In another embodiment, such measurement may be considered in combination with other retinal parameters indicating cellular pathologies associated with neuronal degeneration. Such retinal parameters may include thickness of retinal nerve fiber layer, the diameter of retinal blood vessel, and/or retinal blood flow rate.

Another aspect of the present invention is directed to methods for diagnosing or determining the stage of a neuronal degeneration disease in a patient by employing the pathological changes in the retinal tissue. In one embodiment of this aspect of the invention, inventors disclose methods of diagnosing a patient comprising the steps of (a) imaging the patient's retina with an optical imaging or non-imaging system; (b) detecting a biomarker of the neuronal degeneration disease with said system and quantifying the degree of change in the retinal tissue as compared to the patient's own or a population baseline. In at least one embodiment, the method further includes comparing the observed level of biomarker from a patient's retinal assessment against a control baseline, wherein a deviation is an indication of neuronal degeneration.

In at least an alternative embodiment, a method for diagnosing a patient at risk of developing a neuronal degenerative disease are described including the steps of (a) isolating a tissue comprising a plurality of cells from a source, (b) subjecting said tissue to a medium comprising an agent capable of inducing cellular pathologies consistent with the pathologies in a patient suffering from said neuronal degenerative disease, (c) allowing sufficient contact time between said tissue and said medium, (d) identifying at least one cellular pathology present in patients suffering from said neuronal degenerative disease, (e) assigning a measurement to said identified pathology thereby correlating the severity of the neuronal degenerative disease in said patient.

This methodology may be of particular importance for person who may be at risk of developing or even carrying certain neuronal degeneration disease but are yet symptoms free. As such, the presently described method can be used as screening methodologies among healthcare professionals to ascertain patient's risk of developing the neuronal degeneration disease. In another embodiment, the methods of the present claims can further be employed to monitor therapeutic outcome and the progress of a subjects undergoing AD treatment. In yet another embodiment, the methods of the present claims can further be employed to optimize patient specific drug treatment.

In some embodiments, the neurodegenerative disorder is Alzheimer's Disease (AD). In some embodiments, the AD is Early-Stage AD. AD is presented upon a clinical continuum that comprises preclinical stages, mild-cognitive impairment (MCI) stages, and full dementia. Early-Stage AD as defined herein comprises the pre-clinical and MCI stages of AD. Pathological changes linked to AD, such as those associated with Early-Stage AD, are known to precede overt clinical symptoms for up to a decade prior to clinical diagnosis of AD. There is evidence as early as the preclinical stage of AD of biomarker evidence such as low Aβ₄₂ serum levels, elevated CSF tau or phospho-tau, hypometabolism, cortical thinning/grey matter loss, as well as evidence of some subtle cognitive decline that does not arise to MCI. One point of agreement is that, in a high percentage of those afflicted, AD-related pathological changes begin in the brain 8-10 years before emergence of telltale symptoms. This makes it difficult to identify AD patients at Early-Stage AD, at a time when treatments are most likely to be most beneficial. It is known that, in roughly 60% of all patients that come to see their doctor for the first time with MCI, the symptoms are actually caused by Early Stages of ongoing AD pathology; the remaining 40% are due to other factors such as side-effects of new medications, depression or poor vascular perfusion of the brain. For physicians to properly treat their patients, it is essential for them to know the exact cause of their MCI. The purpose of this invention is to provide a means for physicians to make this distinction and to identify individuals whose MCI is due to an early stage of AD pathology. The pathology of MCI represents a critical area of research, as early detection and diagnosis of AD can lead to a better prognosis. The methods disclosed herein may have particular utility in that they are capable of detecting AD, including Early-Stage AD, and thus allow for appropriate therapeutic treatment to begin which may lead to a better patient outcome. Accordingly, in some embodiments, the present invention is directed to therapeutic treatments to treat a neurodegenerative disease, e.g. AD, including but not limited to Early-Stage AD.

In one aspect of the present invention, the biomarker is a GFAP or MAP2 and the comparison can include an up-regulation of GFAP and/or a down-regulation of MAP2. In at least one embodiment, the optical imaging system may include such systems as described in optical coherence tomography or functional MRI. In such embodiment, the method imaging system may further include administering a detectable contrast agent or a fluorescent marker to the patient. In at least one embodiment, the contrast agent may be a curcumin or a curcumin analog, probe or marker that is administered orally, topically or intervenously to the subject, allowing the stain to bind to the biomarker, then imaging the subject's retinal with for example curcumin imaging devices, autofluorescence, multi-spectral imaging, hyperspectral imaging, fluorescein angiography, ICG angiography and/or optical coherence tomography.

In another embodiment, the method imaging system may include performing large field imaging of retina using retinal imaging light with sufficient depth resolution to ensure detection of the cellular pathology in patient's risk of developing the neuronal degeneration disease.

In yet another embodiment, the optical system may be a non-imaging technique employed by a healthcare professional to assess the integrity of retinal functionality. Such non-imaging techniques may include contrast sensitivity tests such as the Hamilton-Veale test.

In at least one embodiment, optical characterization of retinal tissue signifies the extent and progression of the neuronal degeneration disease, such as ALS, PD, AD, and HD. In at least one preferred embodiment, the neuronal degeneration disease is Alzheimer's disease. In yet another embodiment, the mammal is a transgenic mouse. In yet another embodiment, a change of +10% in inner plexiform layer is an indication of the neuronal disease. In yet another embodiment, a change of −10% in ganglion cell layer is an indication of the stage of Alzheimer's disease.

In another aspect of the present invention, methods of identifying potential drug candidates for treatment of a neuronal degeneration disease including the steps of (a) incubating a tissue slice culture or cells derived such tissue slice culture in a suitable medium, (b) contacting a test compound with a test slice culture or the incubated cells obtained therefrom, (c) allowing sufficient time for cellular absorption of the test compound, (d) assessing the degree of reversal, inhibition or induction of the expression of one or more biomarkers related to a neuronal degeneration disease. In at least one embodiment, the method may further include the step of comparing the measurement with a control slice culture to evaluate the therapeutic potential of the compound for treating or preventing the progression of a neuronal degeneration disease. In another embodiment, the slice culture is derived from a mammal selected from the group consisting of rats, rabbits, guinea pigs and mice. In at least one embodiment, sufficient time for cellular absorption may range from seconds to hours or days, including for example, about 10 seconds, 30 seconds, 60 seconds, 5 minutes, 10 minutes, 30 minutes, 60 minutes, 90 minutes, 120 minutes, more than 3 hours, and more than 24 hours.

In an alternative aspect of the invention, a method for identifying a candidate compound for treatment of a patient at risk of developing a neuronal degenerative disease is described including the steps of (a) isolating a tissue comprising a plurality of cells from a source, (b) subjecting said tissue to a medium comprising an agent capable of inducing cellular pathologies consistent with the pathologies in a patient suffering from said neuronal degenerative disease, (c) allowing sufficient contact time between said tissue and said medium, (d) identifying at least one cellular pathology present in patients suffering from said neuronal degenerative disease, (e) assigning a measurement to said identified pathology thereby correlating the severity of the neuronal degenerative disease in said patient (f) exposing said tissue to a test compound, (g) measuring the reversal, or the inhibition of the cellular pathology identified in step (e).

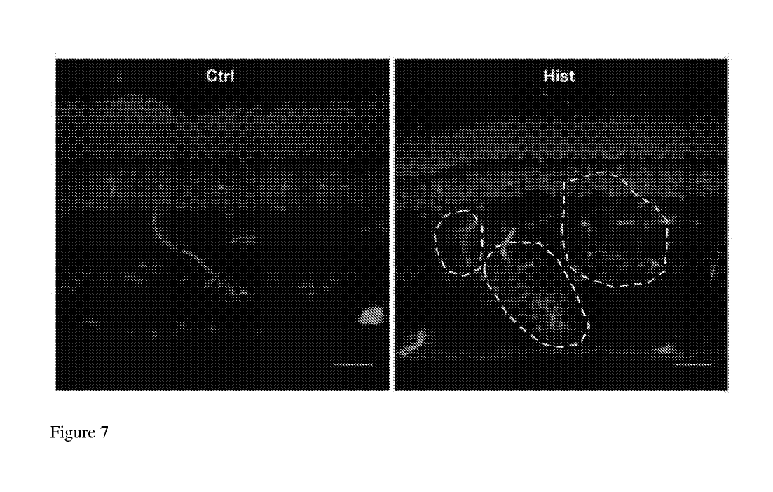

In one embodiment of the present invention, retinal slice cultures treated with histamine display pathologies consistent with AD. In another embodiment changes in tissue characteristics may be observed including leaky blood vessels, change in thickness of a cellular layer, change in vascularization of a cellular layer, changes in dimensions such as length, thickness, area, etc and in appearance such as organization, distribution and degeneration. As shown in FIG. 7-9, treatment of retinal slice cultures with histamine cause BBB breakdown and expression of GFAP and MAP2. In at least one embodiment, in order to test for evidence of BBB breakdown, astrocyte activation, and/or other types of neuronal damage are identified by antibodies against mouse IgG, GFAP, and vimentin. According to this embodiment, the specificity of each antibody in both histamine-treated brains slices as well as control samples can identify extent of retinal damage and/or indicate the prognostic stage of the neuronal disease.

Another feature of the slice culture system of the present invention lies in the changes of several cell types of the culture due to the deep penetrance of the agent such as histamine in the tissue. The thickness of histamine-induced pathology in the slice culture also depends on the concentration of the agent and the length in time for treatment. In exemplary embodiments, the thickness of an affected tissue can range between 1-10 mm, more specifically 1, 2, 2.5, 3, 3.5, 4, 4.5, 5, 5.5, 6, 6.5, 7, 7.5, 8, 8.5, 9 or 10 mm.

In some embodiments, the slice culture is derived from mammals including for example rats, rabbits, guinea pigs and mice. For example, the system of the present invention may include mouse brain organotypic slice cultures (MBO). The mammal used as a tissue source can be a wild-type mammal or can be a mammal that has been altered genetically to contain and express an introduced gene. In some embodiments, the slice culture is derived from human.

The slice cultures of the present invention can also be incorporated into a kit. Additional components of the kit may include for example, antibodies, fluorescent markers, probes, detecting devices such as imaging instruments, one or more agents for inducing desirable pathologies of neuronal degeneration diseases. Further, the kit can include a slice culture for testing as well as a control slice culture.

Another aspect of the present invention provides a method of preparing the above described slice cultures. The method includes treating an organotypic brain slice culture and retinal slice culture with an agent in an amount sufficient to induce pathological changes as in neuronal degeneration diseases. Examples of neuronal degeneration diseases include for example ALS, Parkinson's disease, Alzheimer's disease, and Huntington's disease. A preferred agent is histamine or its analogs or derivatives. The time for treatment ranges from seconds to hours or days, including for example, about 10 seconds, 30 seconds, 60 seconds, 5 minutes, 10 minutes, 30 minutes, 60 minutes, 90 minutes, 120 minutes, more than 3 hours, and more than 24 hours.

Various methods for the preparation of slice culture are known in the art, including for example, U.S. Pat. No. 6,221,670, the entire disclosure of which is hereby incorporated by reference. Cells in slice cultures of the present invention are preferably not only capable of being damaged by histamine, but also of responding to that damage; i.e. astrocytes respond to inflammatory damage by undergoing gliosis and neurons respond to damage by upregulating vimentin production. Therefore, the slice culture system provides a useful model to not only study the effects of inflammatory damage as seen in neuronal degeneration diseases (e.g. AD), but also to study the way the brain responds to this damage.

In an alternative embodiment, the disclosed methods includes the steps of: (a) contacting a test compound with a test slice culture which can be an organotypic brain slice culture or a retinal slice culture; (b) measuring one or more targeted biomarkers correlated with a neuronal degeneration disease; (c) measuring the same biomarkers in a control slice culture; and (d) comparing the measurement from the test slice culture with the measurement from the control slice culture to identify a therapeutic agent, wherein the test slice culture can be treated with an agent to induce pathological changes before, after, or at the same time of contacting with the test compound.

In exemplary embodiments, an organotypic slice culture is typically transferred to a culture dish with media. The culture media can either have a test compound present prior to the introduction of the slice culture or a test compound can be added to the media after the slice culture has been place in the culture dish. A test compound may be dissolved in appropriate vehicle, such as, but not limited to, DMSO, water, physiological saline, or media, to make a stock solution and then diluted into the media.

In one embodiment, the dose range of test compounds to be tested includes for example from about 1 nM to about 100 mg. In at least one embodiment, the compound is applied to the slice cultures for about 1 hours to about 21 days, from about 1 day to about 6 weeks, or from 1 week to 10 weeks. In the case of long term application, fresh media containing compound can be applied periodically; more frequently if rapid loss of compound due to chemical conversion or to metabolism is suspected. One of ordinary skill in the art may adjust the dosage, concentration, frequency, length of time for contacting the test compound with the test slice culture in view of factors such as the specific compound structure, the pre-selected biomarker and the detection sensitivity. In one embodiment, a range or batteries of compounds are tested.

Antibodies suitable for use in the context of this invention include those reported in for example, Levin et al., Brain Res. 1298, 194-207, 2009; Clifford, et al., Brain Res. 1142, 223-36, 2007; Nagele, et al., Neurobiol Aging. 25, 663-74, 2004. The antibodies can be similarly used in measuring the biomarkers in controls.

Another aspect of the present invention provides a method of diagnosing a neuronal degeneration disease in a person in need, comprising: imaging the patient's retina with an optical imaging system; detecting a biomarker of the neuronal degeneration disease with an imaging system and obtaining a reading of the biomarker; comparing the biomarker with a control, wherein a deviation is an indication of neuronal degeneration.

In other embodiments, the disclosed methods relate to a method of treating a patient having Alzheimer's Disease or a neurodegenerative disorder after diagnosing the patient as having Alzheimer's Disease according to any of the diagnostic methods disclosed herein. Many treatments for Alzheimer's Disease are known in the art, but many more therapies are always becoming available. Drugs used to treat cognitive symptoms generally fall into two classes, cholinesterase inhibitors and memantine. Cholinesterase inhibitors increase available levels of the neurotransmitter acetylcholine in the brain, which has been shown to be depleted in the brains of those suffering from AD. Cholinesterase inhibitors can also improve neuropsychiatric symptoms, such as agitation or depression. Examples of cholinesterase inhibitors include donepezil, galantamine, and rivastigmine. Donepezil is one of the most common treatments for AD, and is the only FDA treatment approved for all stages of AD, including Early-Stage AD. Side effects of cholinesterase inhibitors are often modest, except in those who have cardiac conduction disorders, in which case serious side effects may occur. Memantine works on the glutamatergic system by blocking NMDA receptors. Antidepressants may be prescribed to help control the behavioral symptoms that are associated with AD, as well as anti-anxiety medications such as benzodiazepines, however these may in some cases actually increase the severity of some side effects of AD and so are not prescribed as often.

Due to its increasing prevalence, AD has been a target for experimental procedures. Many of the new treatments are directed to targeting beta-amyloid plaques. For example, monoclonal antibodies (mAbs) have been generated to targeted beta-amyloid, particularly solanezumab and aducanumab. Saracatinib is a drug which targets Fyn, which is over-activated when combined with beta-amyloid, and may trigger more rapid neurodegeneration. There are several experimental drugs which seek to block the activity of enzymes that form beta-amyloid, as well as tau aggregation inhibitors. There are also drugs that seek to combat inflammation, as many individuals view inflammation as a key cause for the symptoms of AD, although there is disagreement on this topic in the scientific community.

There are a number of other experimental procedures out there for the treatment of AD, including Early-Stage AD, such as deep brain stimulation (DBS). One surprising method of treatment has been through sonication, although this has only been recently reported to work in mice, but would still be considered to be covered by this invention.

The examples set forth below also serve to provide further appreciation of the disclosed invention, but are not meant in any way to restrict the scope of the invention.

### EXAMPLES

### Example 1

### Experimental Procedures

### Antibodies

Human IgG antibodies (polyclonal, Cat. No. BA-3000, dilution=1:2000) and mouse IgG antibodies (polyclonal, Cat. No. BA-9200, dilution=1:2000) were obtained from Vectastain (Foster City, Calif.). GFAP antibodies were obtained from Millipore (Billerica, Mass.) (polyclonal, Cat. No. AB5804, dilution=1:1000). Vimentin antibodies were obtained from Sigma (Saint Louis, Mo.) (monoclonal, Cat. No. V6630, dilution=1:200). The specificity of each of these antibodies was confirmed via western blot or ELISA (data not shown).

### Animals

C57BL/6J mice were obtained from Jackson Laboratories (Bar Harbor, Me.) and used at 9 months of age. Mice were maintained on ad libitum food and water with 12-hour light/dark cycle in an AAALAC-accredited vivarium under a UMDNJ IACUC-approved protocol.

### Primary Mouse Brain Organotypic Cultures

Primary mouse brain organotypic cultures were prepared as described previously ((Levin et al., Brain Res. 1298, 194-207, 2009). Briefly, the brains were removed from C57BL/6 mice (n=8) and cut to either 1 mm or 2 mm thickness using a tissue chopper. The brain slices were then placed in medium containing 25% inactivated horse serum, 25% Hanks' BSS, 50% DMEM, and 25 mg/l penicillin-streptomycin (Invitrogen, Carlsbad, Calif.) in 6-well culture dishes and maintained for 30 min at room temperature. The brain slices were then moved to either fresh media (control) or fresh media containing 450 μM histamine (Sigma, Saint Louis, Mo., Cat. No. H7125-1G) in 6-well culture dishes for 1 hr at 37° C. in a 5% CO2-enriched atmosphere. The brain slices were fixed with 4% paraformaldehyde (PFA) in PBS at room temperature and processed for immunohistochemistry as described below.

### Human Brain Tissue

Brain tissue from patients with sporadic AD (n=21) and age-matched, neurologically normal individuals (n=13) were obtained from the Harvard Brain Tissue Resource Center (Belmont, Mass.), the Cooperative Human Tissue Network (Philadelphia, Pa.), the UCLA Tissue Resource Center (Los Angeles, Calif.) and Slidomics (Cherry Hill, N.J.). Post-mortem intervals were <24 h and pathological confirmation of AD was evaluated according to the criteria defined by the National Institute on Aging and the Reagan Institute Working Group on Diagnostic Criteria for the Neuropathological Assessment of AD (Hyman and Trojanowski, 1997). AD tissues displayed amyloid plaques and neurofibrillary tangles, and control tissues exhibited no gross pathology and minimal localized microscopic AD-like neuropathology. Tissues were processed for routine paraffin embedding and sectioning according to established protocols. All human brain tissue was used with prior approval from IRB.

### Immunohistochemistry

The mouse brain organotypic brain slices were stored in 4% PFA overnight at 4° C. The brain slices were then infiltrated with 10% sucrose in PBS for 2 hours, followed by 30% sucrose in PBS overnight at 4° C. under constant, gentle agitation. Using a Leica cryostat, 12 μm thick frozen sections were cut, mounted onto Fisher Super Frost Plus slides, and air dried. Immunohistochemistry for the paraffin-embedded tissues was carried out using procedure well known in the art. Briefly, tissues were deparaffinized using xylene and then rehydrated through a graded series of decreasing concentrations of ethanol. Next, protein antigenicity was enhanced by microwaving sections in citrate buffer. The paraffin-embedded tissues were then processed in the same way as the frozen sections described below.

Immunohistochemistry for the frozen sections was carried out as previously described ((Levin et al., Brain Res. 1298, 194-207, 2009). Briefly, tissues were rehydrated with PBS for 2 min. The endogenous peroxidase was quenched by treating sections with 0.3% _{H202} for 30 min. First, sections were incubated in blocking serum for 30 min. at room temperature, and then treated with primary antibodies at appropriate dilutions for 1 hr at room temperature. Next, sections were thoroughly rinsed with PBS, and sections were incubated with biotin-labeled secondary antibody for 30 min. at room temperature. Sections were then treated with the avidin-peroxidase-labeled biotin complex (ABC, Vector Labs, Foster City, Calif.) and visualized by treating with either 3-3-diaminobenzidine-4-HCL (DAB)/H2O2 (Biomeda, Foster City, Calif.) or NovaRed (Vector Labs, Foster City, Calif., Cat. No. SK-4800). Sections were then lightly counterstained with hematoxylin, dehydrated through increasing concentrations of ethanol, cleared in xylene and mounted in Permount. Specimens were examined and photographed with a Nikon FXA microscope, and digital images were recorded using a Nikon DXM1200F digital camera and processed using Image Pro Plus (Phase 3 Imaging, Glen Mills, Pa.) image software.

### Quantitation

Leaky and non-leaky vessels, GFAP-positive and -negative astrocytes, and vimentin-positive and -negative neurons were counted in sections of primary mouse brain organotypic culture slices. Images were optimized and counting was performed using the counting feature of Adobe Photoshop CS3. Three sections were examined per treatment group, with at least ten viewing fields counted from each section, for a total of 30 viewing fields per treatment group. Only blood vessels with endothelial cell nuclei, astrocytes with their nuclei, and neurons with their nuclei within the plane of section were included in the count. Blood vessels were considered leaky if they showed a gradient of immunostaining surrounding the vessel. Astrocytes were considered GFAP-positive if they showed staining within their cell body and dendrites. Neurons were considered vimentin-positive if they showed immunostaining in the cell body and/or main apical dendrite. The percentage of total leaky vessels, GFAP-positive astrocytes, and vimentin-positive neurons for histamine treated and control slices were determined.

### Analysis

Immunohistochemistry (IHC) was used to evaluate the effects of histamine on BBB permeability and the response of cells within the surrounding brain parenchyma. Antibodies against mouse IgG, GFAP, and vimentin were used in order to test for BBB breakdown, astrocyte activation, and neuronal damage-response, respectively, in histamine-treated brains slices as well as controls.

### Pathological Features Seen in AD Include BBB Breakdown, Activation of Astrocytes, and Neuronal Expression of Vimentin

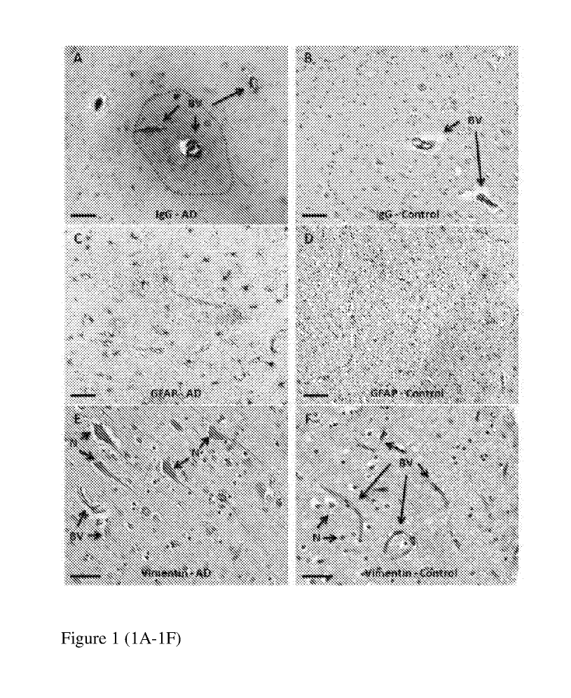

In AD brains, the permeable status of the BBB can be revealed by immunostaining for proteins such as IgG that are normally confined to blood vessels with an intact BBB. For example, in AD brains, extravasated IgG is often localized to a perivascular leak cloud emerging from a discrete region along the vessel) or more global (present along a much greater length of the vessels) (FIG. 1A). Conversely, in aged-matched, neurologically normal control brains, such perivascular leak clouds are rarely encountered and IgG is restricted to the lumen of blood vessels (FIG. 1B). AD brains show a marked increase in activated astrocytes (FIG. 1C), as determined by an increased intensity of GFAP-positive immunostaining compared to control brains (FIG. 1D). The cell bodies and apical dendrites of neurons within areas of pathology in AD brains are selectively vimentin-positive (FIG. 1E), whereas vimentin expression is generally restricted to vascular endothelial cells in control brains (FIG. 1F).

### Histamine Causes BBB Breakdown in MBOs.

A major pathology consistently associated with AD is breakdown of the BBB. To investigate the effect of histamine on BBB permeability in vitro, MBOs were treated with 450 μM histamine. In order to test the penetrance of histamine, treatments were independently carried out on brain slices at two different thicknesses: 1 mm and 2 mm. The slices were individually processed and sectioned. Comparable results were obtained with both sets, indicating that histamine penetrance under experimental conditions was complete in the 2 mm slices. The 2 mm slices were sectioned at different depths from the surface (Proximal=0-350 μm, Middle=350-700 μm, Distal=700-1050 μm). The results obtained from these sections are presented below. Those obtained from the 1 mm slices are provided as supplemental information.

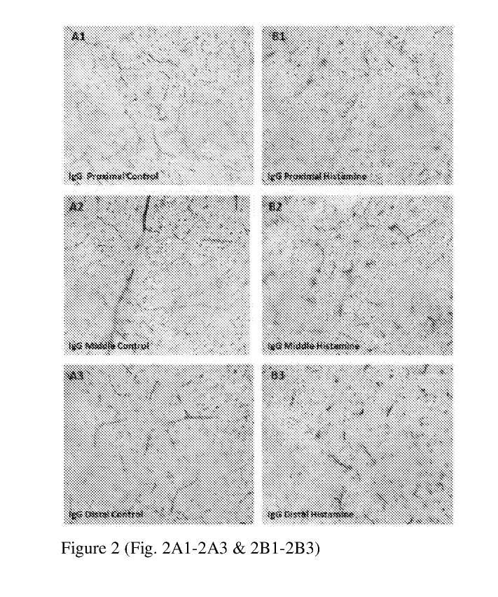

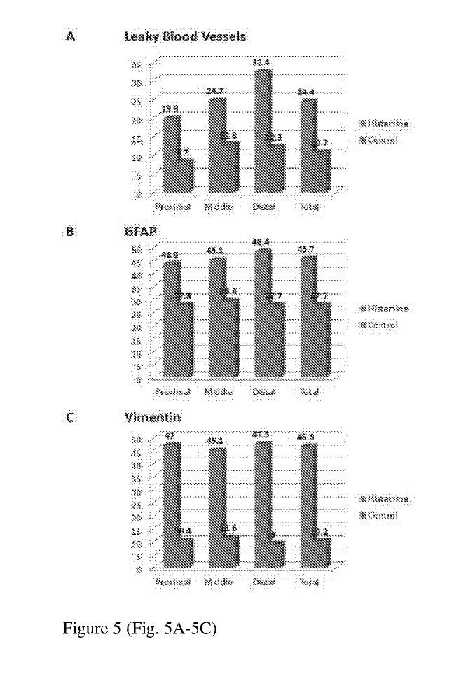

IHC using antibodies against mouse IgG was used to detect IgG in the brain interstitium in sections of MBOs as evidence of BBB permeability. In histamine treated MBOs, blood vessels showing perivascular leak clouds (FIG. 2B1, 2B2, and 2B3), were far more numerous than in corresponding sections of untreated control MBOs (FIG. 2A1, 2A2, and 2A3), regardless of their location within the tissue. IgG extravasiated out of blood vessels more often in histamine treated sections obtained from the proximal portion of the tissue (19.9% in histamine treated vs. 8.2% in control), the middle portion of the tissue (24.7% histamine treated vs. 12.8 control), and the distal portion of the tissue (32.4% histamine treated vs. 12.3% control). Overall, histamine treated MBOs showed evidence of perivascular leakage more than two-fold as often when compared to controls (FIG. 5A).

It has previously been shown that in dynamic systems, histamine stresses the tight junctions between adjacent endothelial cells, creating intercellular space that allows for the leakage of humeral elements into the surrounding parenchyma (Kumar et al., 2009; Majno et al., 1969; van Hinsbergh and van Nieuw Amerongen, 2002). BBB breakdown is an important pathology commonly associated with AD, as it allows for the extravasation of potentially damaging humoral elements such as IgG, complement components, Aβ42, and proinflammatory mediators. In our study we showed that histamine had a similar effect on the BBB in MBOs, leading to extravasation of serum components as indicated by IgG leakage. One way histamine may be exerting its effects on the endothelial cells of the BBB is through calcium (Ca²⁺). Histamine has been previously shown to cause significant increases in intracellular Ca²⁺ in endothelial cells. This dysregulation of Ca²⁺ causes cellular contraction, leading to increases in the permeability of the BBB. Additionally, exogenous histamine has been shown to induce changes in endothelial cells whether applied luminally or abluminally. Ca²⁺ dysregulation also plays an important role in AD pathology, where it may lead to a variety of changes including cellular loss. It has also been shown that mouse models of AD display alterations in Ca²⁺ regulation.

### Histamine Treatment Leads to Astrocyte Activation in MBOs.

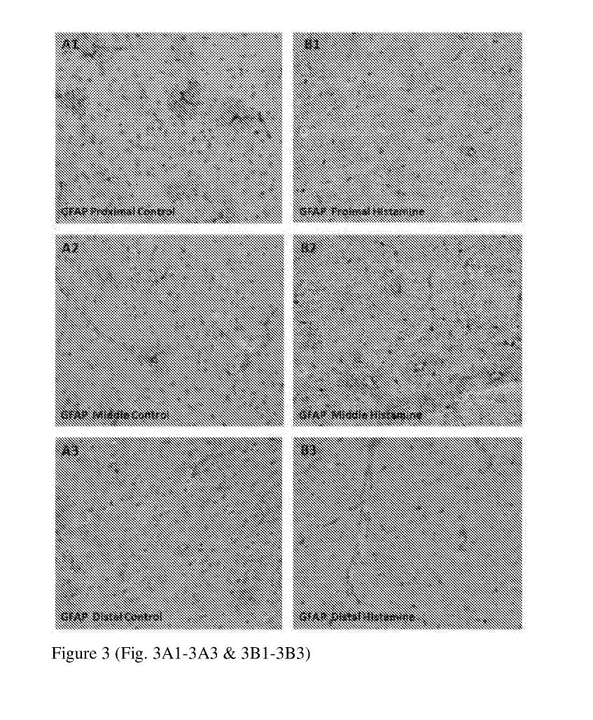

Increased gliosis in regions of pathology is observed in the brains of both AD patients (Mancardi et al., 1983; Simpson et al., 2010; Wharton et al., 2009) and the triple-transgenic mouse model of AD {Olabarria, 2010 #252}. We have investigated the effects of histamine on astrocytes resident in MBOs using their relative levels of GFAP expression as an indicator of their activation. Immunohistochemical staining of MBOs with and without histamine treatment using anti-GFAP antibodies was carried out and the results are presented in FIG. 3. An increased number of GFAP positive cells were observed in sections from histamine-treated MBOs (FIGS. 3B1, 3B2, and 3B3) versus control (FIGS. 3A1, 3A2, and 3A3), regardless of their location within the tissue. Quantitative analysis revealed that histamine treatment resulted in an increase in GFAP expression in the proximal (43.6% histamine treated vs. 27.8% control), middle (45.1% histamine treated vs. 29.4% control), and distal (48.4% histamine treated vs. 27.7% control) portions of the MBOs. This increase was nearly two-fold in total (FIG. 5B).

Inflammation within the brain parenchyma contributes significantly to AD pathogenesis, as was extensively reviewed by Akiyama et al. A good measure of this inflammation is astrocyte activation, also known as gliosis, as observed through upregulation of GFAP by astrocytes. Activated astrocytes are evident both in transgenic animal models of AD as well as in the brains of AD patients in regions surrounding amyloid plaques. In the present study, we showed that histamine is able to increase gliosis in MBOs almost two-fold. It is possible that this increase is due to a direct interaction between histamine and astrocytes or, alternatively, that the histamine-induced increase in BBB permeability allows other inflammatory mediators access to the normally privileged brain parenchyma. In either case, histamine-treated MBOs show significant inflammation as measured by increased gliosis, a pathology common to AD.

Since it is known that astrocytes respond to BBB breakdown by a swelling of their foot processes, it is possible that the gliosis seen in our present study is an extension of the histamine-induced BBB breach. Furthermore, astrocytes respond to damage within the brain via activation. Once activated, the astrocytes may begin the process of removing damaged proteins and debris associated with cellular death, a consequence of the initial damage. The fact that astrocytes in our current study also become activated indicates that pathological changes downstream to BBB breakdown are effectively functioning in MBOs, thus speaking to the power of our model system in recapitulating pathologies seen in living patients.

### Vimentin is Expressed in Neurons in Response to Exposure to Histamine.

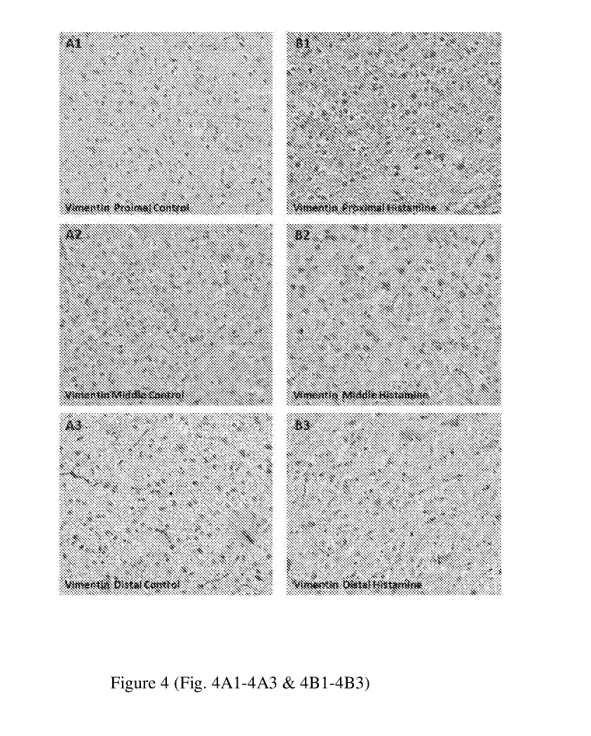

Under normal, non-pathological conditions, vimentin is expressed in the brain by endothelial cells and developing neurons, but not by mature neurons in adult brains. In AD and traumatic injury neurons can undergo a localized damage-response that includes the expression of vimentin in an attempt to reestablish their dendritic trees. In order to test the effects of histamine on the structural and functional integrity of neurons, histological sections of MBOs with and without histamine treatment were immunostained using antibodies specific for vimentin Histamine treatment was accompanied by increased vimentin expression within neurons of sections treated with histamine (FIGS. 4B1, 4B2, and 4B3) when compared to control sections (FIGS. 4A1, 4A2, and 4A3). This increase in vimentin expression holds true regardless of the location within the tissue. Images from these samples were quantitated to determine the proportion of vimentin positive neurons in histamine-treated versus control brain slices. MBOs treated with histamine showed a greater than four-fold increase in neurons expressing vimentin when compared to control sections that were not treated with histamine (FIG. 5C). This held true for neurons in the proximal (47% histamine treated vs. 10.4% control), middle (45.1% histamine treated vs. 11.6% control), and distal (47.5% histamine treated vs. 9% control) portions of the MBOs.

Vimentin is an intermediate filament protein that is important for neuronal growth and development and is necessary for the extension and branching of neurites. As a result, it is commonly expressed by neuronal precursor cells in the developing CNS of rodents and humans. In the healthy, adult brain, vimentin expression is restricted mainly to endothelial cells. However, in the AD brain, vimentin has also been found within neurofibrillary tangles, a hallmark pathology associated with the disease. More recently, we have shown that vimentin is expressed by neurons in AD brains, possibly as part of a damage-response mechanism in order to reestablish dendritic trees. In our present study, histamine application resulted in significant peri-nuclear vimentin expression within the cell bodies of neurons, whereas it was mainly restricted to the vascular epithelium of control MBOs.

Since neurons throughout the brain express histamine receptors, it is possible that histamine directly binds to neurons in MBOs, causing localized damage due to excitotoxicity and inducing the neuron's damage-response, including expression of vimentin. Alternatively, the BBB breakdown caused by histamine could allow plasma components, such as brain-reactive autoantibodies, complement components, and Aβ42, to directly bind to and damage the MBO neurons, as has been shown in AD brains. Histamine's ability to elicit a neuronal response so quickly in MBOs shows the immediacy of the neuronal damage-response mechanism. Within the relatively brief 1 hour timeframe of histamine exposure in this study, neurons already begin attempting to repair themselves and reestablish their lost connections so that they can continue to function normally. The fact that so many neurons display a damage response in the present study also indicates the overall extent to which histamine can mediate damage in the brain.

### Histamine is Able to Induce AD-Like Pathologies in MBOs.

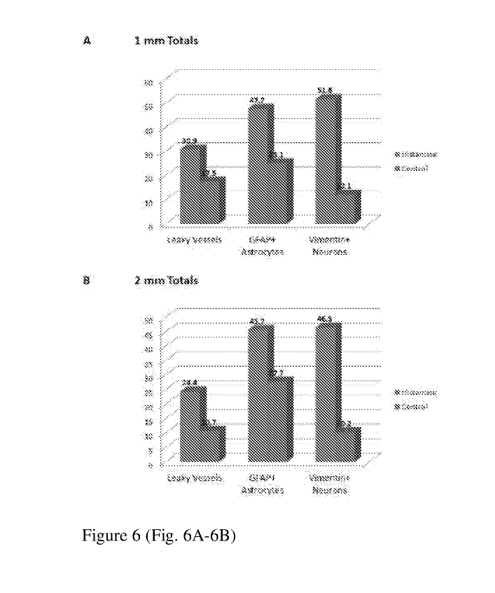

Treating either 1 mm (FIG. 6A) or 2 mm (FIG. 6B) thick MBOs with 450 μM histamine for 1 hour produced similar results. Within the 1 mm MBOs, histamine treatment caused increased blood vessel leakage (30.9% histamine vs. 17.5% control), astrocyte activation (47.7% histamine treated vs. 25.1% control), and neuronal damage-responses (51.8% histamine treated vs. 12.1% control), when compared to control-treated cultures. 2 mm thick MBOs treated with histamine also showed increases in AD like pathology including blood vessel leakage (24.4% histamine treated vs. 10.7% control), increased GFAP expression by astrocytes (45.7% histamine treated vs. 27.7% control), and vimentin expression by neurons (46.5% histamine treated vs. 10.2% control). Taken together, our data indicates that histamine is able to readily penetrate and permeate the brain interstitium in a relatively short period of time and in doing so, can induce some of the cellular pathologies associated with AD.

As shown in the current study, histamine is able to induce AD-like pathology in MBOs up to 2 mm in thickness. This indicates that histamine's tissue penetrance within the brain is substantial enough to create changes in several cell types of the brain, even in the relatively short (1 hr) treatment time. We have shown histamine to be a powerful molecule in terms of its abilities to create pathological changes in MBO brains that are consistent with those found in AD. This is in line with its proinflammatory nature. Interestingly, it has been previously noted that the use of anti-inflammatory drugs have benefits in treating the cognitive symptoms of AD. On the other hand, whether anti-histamines could be a useful avenue of treatment may depend on the time of administration since the cellular changes found in AD have been shown to predate the symptomology by years to decades.

It is entirely possible that histamine-induced inflammation is an early and/or downstream contributor, in AD. After all, histamine is capable of creating several pathologies consistent with the disease process. Increases in histamine are capable of triggering a cascade of problems, starting with the BBB breakdown that allows Aβ42 and other humeral element access to cells of the brain. Once histamine enters the brain parenchyma, it could potentate these adverse effects by damaging neurons, thus resulting in increases in gliosis.

Cells in MBOs are not only capable of being damaged by histamine, but also of responding to that damage; i.e. astrocytes respond to inflammatory damage by undergoing gliosis and neurons respond to damage by upregulating vimentin production. As such, our histamine-treated MBO model system provides a useful model to not only study the effects of inflammatory damage as seen in AD, but also to study the way the brain responds to this damage. In conclusion, our current study indicates that MBOs treated with histamine are a quick, simple, and effective tool for investigating pathological changes associated with AD.

### Example 2

Retinal slice cultures treated with histamine also display pathologies consistent with AD. As shown in FIG. 7-9, retina with BRB breakdown and expression of GFAP and MAP2 have been observed after treatment of retinal slice cultures with histamine.

As shown in FIG. 7, retina with BRB breakdown and extravasated IgG surrounding blood vessels (indicated by dotted circles) are observed after histamine treatment (Panel: Hist). In untreated retinal slices, IgG is confined to BV lumen (Panel: Ctrl).

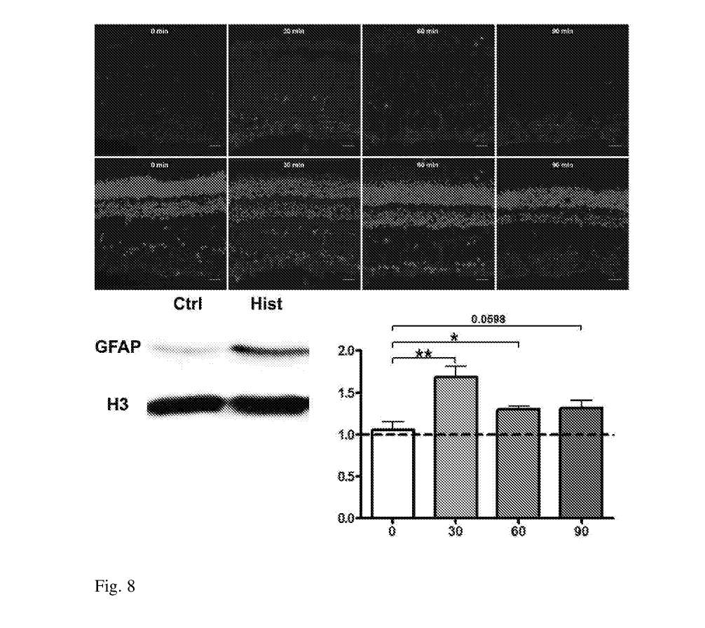

Histamine treatment also increases the expression of GFAP in retinal slices. FIG. 8 illustrates retinal slice cultures after treated for indicated time with histamine (0, 30, 60 or 90 min). The slices were then processed to generate cryosections for immunostaining. The expression of GFAP was monitored by immunostaining. GFAP is observed in green. The results are presented in two rows: the top one without the nuclei and the bottom one—with the nuclei in blue. A structural response is evident. In addition, there is also a change in the expression level of GFAP. The staining has been quantitated by a combination of open source software and added code. The results are presented in histograms. An elevation in GFAP expression is seen. The results were independently confirmed by Western blotting. Total protein was isolated and probed for GFAP levels with histone H3 as a control.

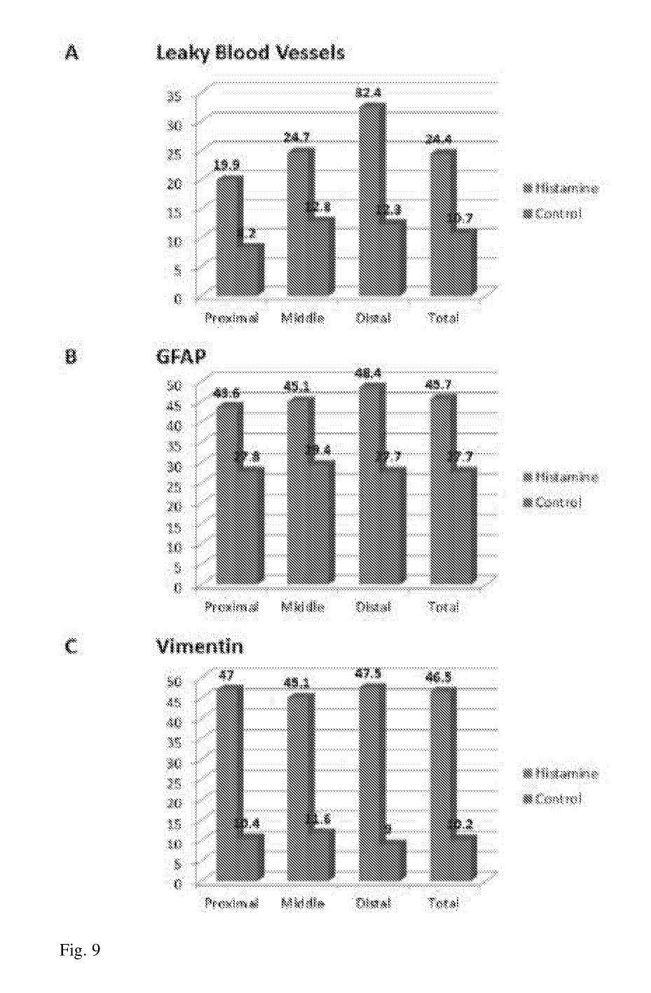

Retinal slices were treated for indicated time with histamine (0, 30, 60 or 90 min) as shown in FIG. 9. The slices were then processed to generate cryosections for immunostaining. The expression of GFAP was monitored by immunostaining. MAP2 is observed in red. The results are presented in two rows: the top one without the nuclei and the bottom one—with the nuclei in blue. A structural response is evident. In addition, there is also a change in the expression level of GFAP. The staining has been quantitated by a combination of open source software and added code. The results are presented in histograms. An decrease in MAP2 expression is seen. The results were independently confirmed by Western blotting. Total protein was isolated and probed for GFAP levels with histone H3 as a control. It is seen that histamine treatment decreases the expression of MAP2.

### Example 3

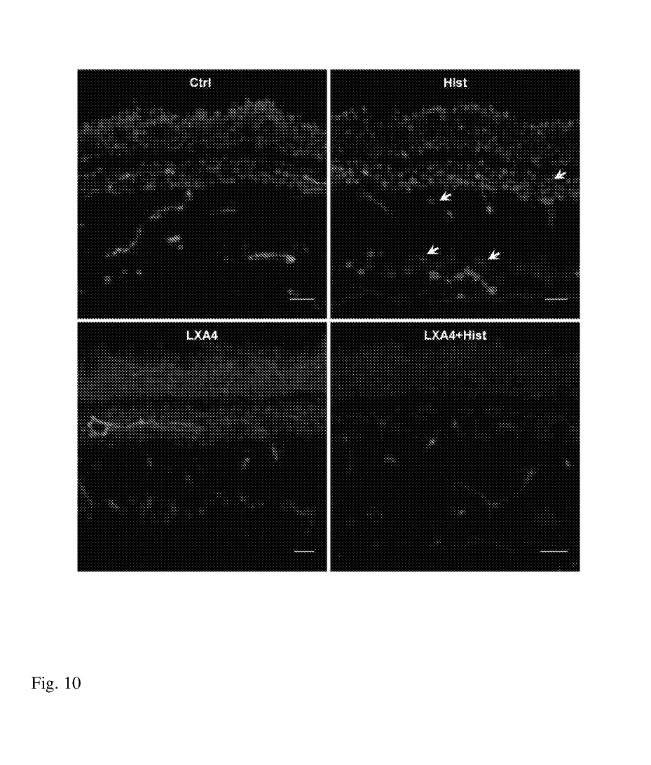

The slice cultures of the present invention can also be used for evaluating or identifying compounds with therapeutic potential for treating or preventing neuronal degeneration diseases such as AD. As shown in FIG. 10, retinal slices were treated with histamine with or without lipoxin A4. Untreated samples served as control. The slices were then processed to generate cryosections for immunostaining. The location of IgG (an indicator of BRB breach) was monitored by immunostaining. IgG was observed in red. The results were presented in two rows: the top one without LXA4 and the bottom one—with LXA4 treatment. In the absence of LXA4, neurons were loaded with IgG and appeared in red (indicated by arrowheads) after histamine treatment. Addition of LXA4, however, conferred protection from these effects.

### Example 4

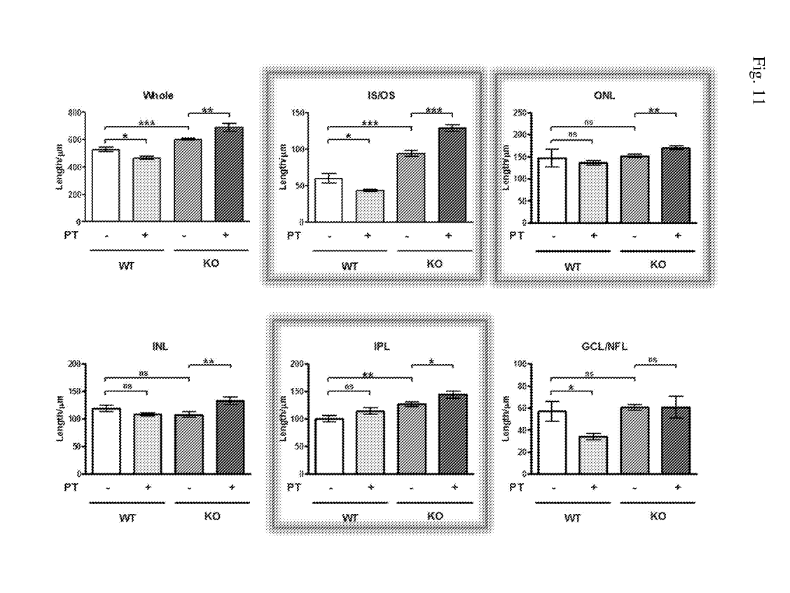

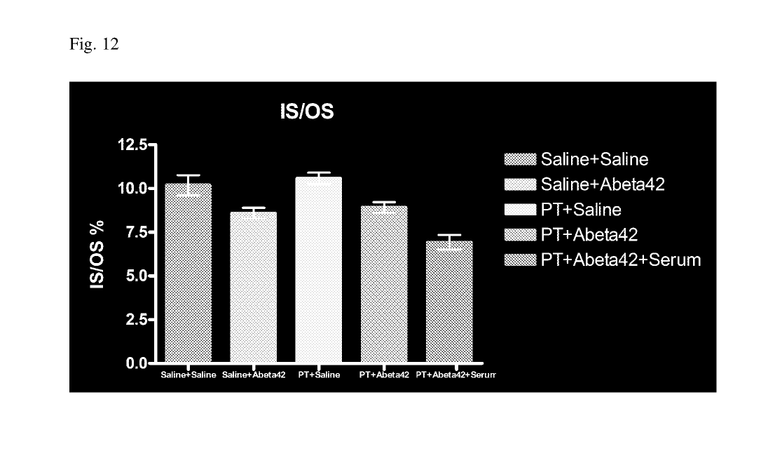

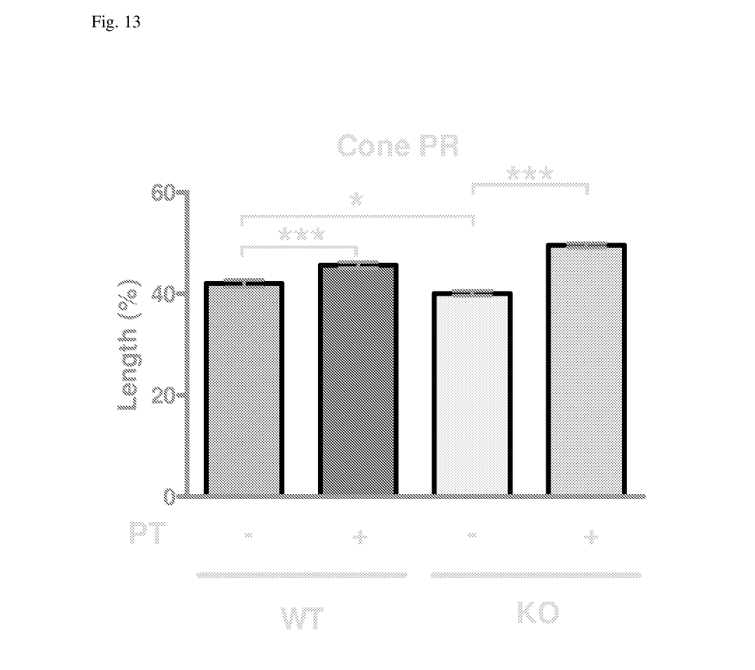

This experiment followed the protocol of Example 1. This example shows how the blood-brain barrier in S100BKO (knockout) mice parallels blood-brain barrier dysfunction. S100BKO mice demonstrate significant BRB compromise and IgG-bound cells in the retina. Appearance of BRB breaches and IgG-bound neurons in the retina of S100BKO mouse brains is age-dependent. Immunostaining of IgG on retinal sections from different age groups (5-, 9- and 18-month old) was carried out. IgG staining was confined to vasculature in 5-month old mice. Leak clouds (marked by dotted circles) and IgG-positive neurons (indicated with arrowheads) appeared at 9 months. When the age reaches 9 months, IgG-positive photoreceptors were observed Different retinal layers were targeted, control, AB42, PT, PT+AB42, or PT+AB42+Serum, with length measures reported in FIG. 12. Similar changes were observed due to the loss of the blood retinal barrier in S100Bko (FIG. 11). Cone photoreceptors are targeted, as shown in FIG. 13.

### Example 5

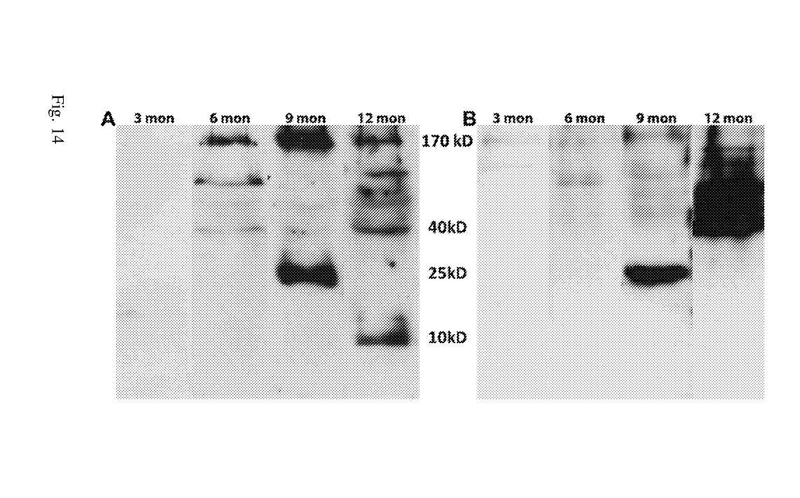

This experiment followed the protocol of Example 1. This example illustrates how retinal antigens are shared with the brain and how it is age-dependent. Western blots of the whole brain protein extract from WT mice were probed with pooled sera from S100BKO mice at 3 (3 mon), 6 (6 mon), 9 (9 mon) or 12 (12 mon) months of age. A representative result is shown in FIG. 14A. Specific bands were observed with sera from 6-months or 9-months old animals, indicating an age-dependent change of the autoantibody profile. (B) Western blots of the swine retina protein extract were probed with pooled sera from S100BKO mice at 3 (3 mon), 6 (6 mon), 9 (9 mon) or 12 (12 mon) months of age. A representative result is shown in FIG. 14B. Similar bands were obtained in both brain and retina at the same age groups, demonstrating a profile shift of autoantibodies upon aging. It was noted that the serum from 12 month old sera reacted with more bands in the retina compared to those in the brain. Pertussis toxin treated wild type mice and S100B KO mice displays retinal neuronal damages shown by SV2 immunostaining and MAP2 immunostaining

### Example 6

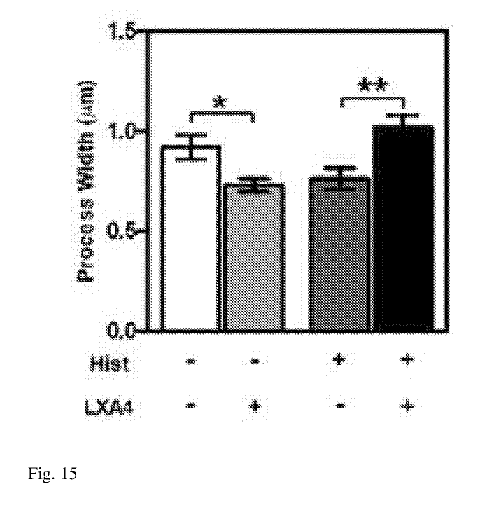

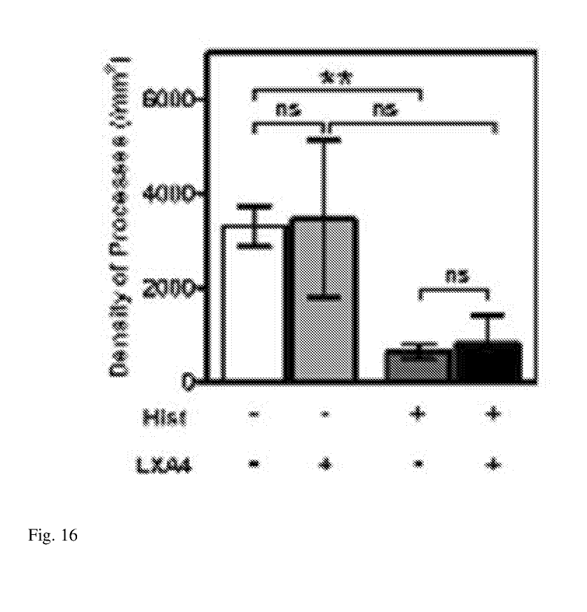

This experiment followed the protocol of Example 1. This example illustrates a new analysis added for drug evaluation in ex vivo retinal culture. Specifically, Muller Cell Processes are Compromised by Histamine and Rescued by LXA4 Treatment. Immunostaining for GFAP was presented from retina which was untreated (damage-induced with histamine only treated with LXA4 only, or exposed to both histamine and LXA4. Positive staining was obtained in the processes of Muller cells across the retina or around the BVs. The width of the Muller cell processes from all groups were presented in FIG. 15. Statistical significance was determined by two-tailed Student's t test. *, P<0.05; **, P<0.01; ***, P<0.001. Mean±SEM is plotted in the graph. Immunostaining against MAP2 was presented retina which was untreated, damage-induced with histamine only, treated with LXA4 only, or exposed to both histamine and LXA4. Positive staining was obtained in the processes and cell bodies of ganglion cells. The density of continuous, MAP2-positive ganglion cell processes from all groups is presented in FIG. 16. Statistical significance is determined by two-tailed Student's t test. *, P<0.05; **, P<0.01; ***, P<0.001. Mean±SEM is plotted in the graph.

### Example 7

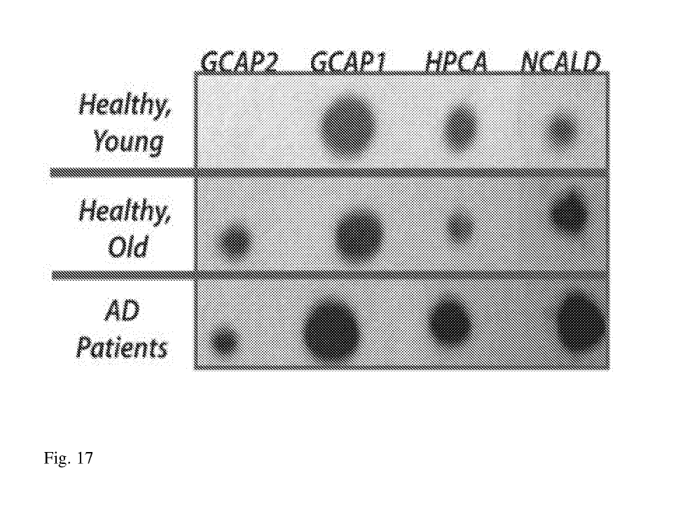

This experiment followed the protocol of Example 1. Sera from control, aged and AD patients recognize NCS proteins diffrentially. All these proteins are expressed in the retina. Specific proteins were targeted by antibodies from AD sera, as shown in FIG. 17.

### Example 8

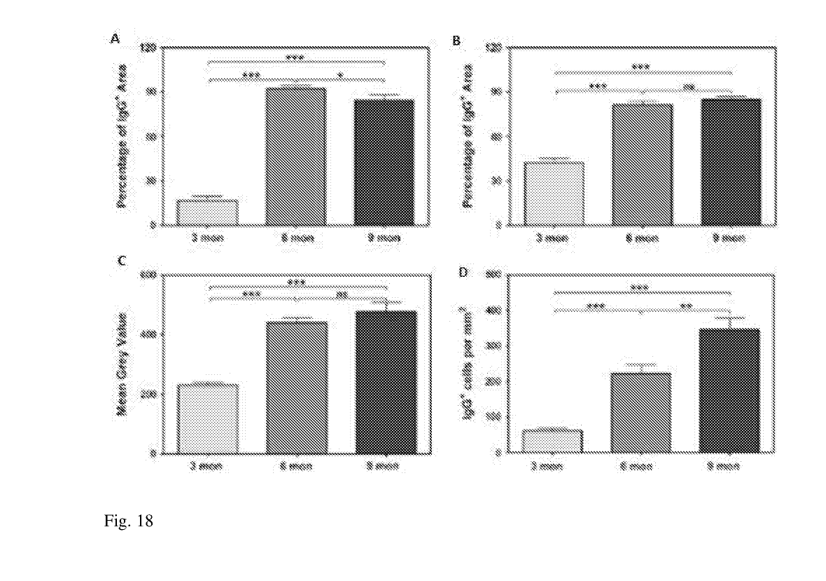

This experiment followed the protocol of Example 1. This experiment involved S100B knockout (KO) mice, focusing on age-dependent increase of blood-brain barrier permeability and neuron-binding autoantibodies in SB100KO mice. S100BKO mice demonstrated significant BBB compromise and IgG-bound cells in the brain. Overlay of IgG immunostaining (red) with DAPI (blue) is presented from cortical region of the brain. (FIG. 18A) In untreated wild type (WT) brains, IgG-positive staining was confined to the microvasculature, indicating intact blood vessels (arrows). (FIG. 18B) With PT treatment, WT brain showed IgG-positive microvascular leaks. (FIG. 18C). In the S100BKO mouse brain (KO), even without PT treatment, IgG-positive leaks (marked by white dotted circle) were observed. (FIG. 18D). Neurons (arrowheads) were intensely bound by IgG in S100BKO mice treated with PT.

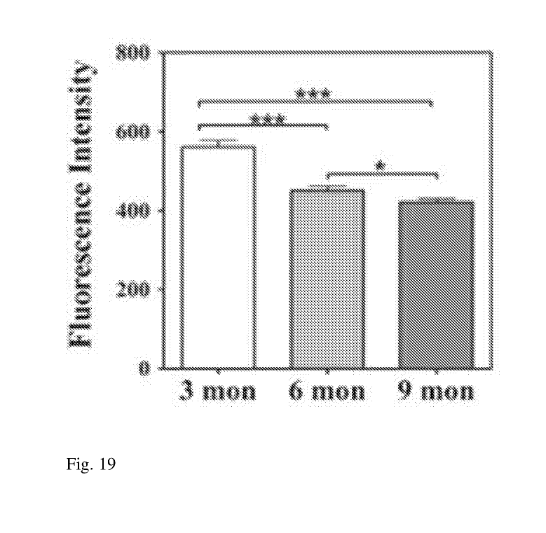

Appearance of BBB breaches and IgG-bound neurons in S100BKO mouse brains is age-dependent. Immunostaining of brain sections from different age groups (3-, 6- and 9-month old) was carried out using fluorescent or chromogenic methods of detection. Similar results were obtained with both methods. IgG staining was confined to vasculature in 3-month old mice. Leak clouds (marked by dotted circles) and Ig-positive neurons (indicated with arrowheads) appeared at 6 months and worsened by 9 months. (FIG. 19). Quantitation of the images also demonstrates an age-dependent BBB breach in the S100BKO mice. 3 fluorescent and 3 chromogenic immunostainings were performed on 4 mice per group. For each sample, 8-18 fields were taken to perform the analysis. The percentages of IgG-positive area, mean grey values, and IgG-positive neurons per square mm, from each group were measured by ImageJ and plotted in a graph. Student t-tests were performed between indicated groups. *, P<0.05; **, P<0.01; ***, P<0.001.

Appearance of brain-reactive autoantibodies from S100BKO mice is age-dependent. Western blots of the whole brain protein extract from WT mice were probed with pooled sera from WT mice (WT) or from S100BKO mice at 3 (3 mon), 6 (6 mon) or 9 (9 mon) months of age. Molecular size markers are indicated alongside. Specific bands were observed with sera from 6-months or 9-months old animals, indicating an age-dependent change of the autoantibody profile. Western blot of the whole brain protein extract from 3- (3 mon), 6- (6 mon) or 9- (9 mon) month old S100BKO mice was probed with pooled sera from 9-month old S100BKO mice. Identical bands were obtained across the age groups, demonstrating an unaltered antigen profile upon aging.

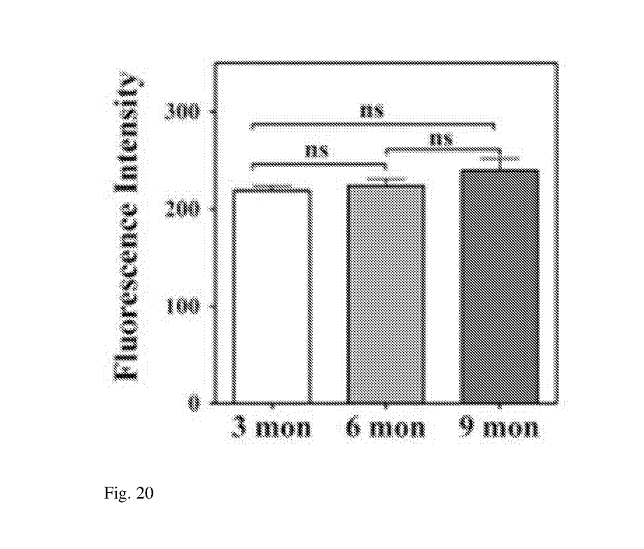

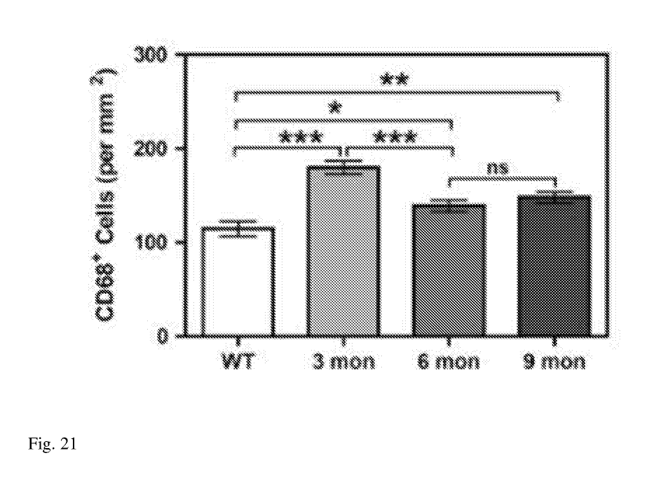

TJ folds in S100BKO mouse brain are disorganized compared to WT. In the S100BKO mice, the TJ folds appeared discontinuous and/or flat while the ridges of BVECs were continuous and well-defined in WT mouse as analyzed by SEM. Neuronal damage was evident upon aging in S100BKO mice. Decreased staining for MAP2 was observed at 6- and 9-month old S100BKO mice. Immunostaining of MAP2 in S100BKO mouse brains from different age groups were performed. The fluorescence intensity (quantified as mean grey values) were measured by ImageJ and plotted in FIG. 20. Student t-tests were performed between indicated groups. *, P<0.05; **, P<0.01; ***, P<0.001. Astrocytic activation is not detectable upon aging in S100BKO mice. No significant change in distribution or intensity of staining for GFAP was detected. Immunostaining of GFAP in S100BKO mouse brains from different age groups was performed. The fluorescence intensity (mean grey values) was measured by ImageJ and plotted in FIG. 21. Student t-tests were performed between indicated groups. *, P<0.05; **, P<0.01; ***, P<0.001. Activated microglia are more abundant in S100BKO mice compared to WT and maximal at 3 months. Increased staining for CD68-positive microglia was observed in S100BKO mice. Immunostaining of CD68-positive microglia in WT and S100BKO mouse brains from different age groups were performed. The number of positive cells per unit area was determined through ImageJ and plotted in FIG. 26. Student t-tests were performed between indicated groups. *, P<0.05; **, P<0.01; ***, P<0.001.

1. A method for diagnosing a patient at risk of developing a neuronal degenerative disease comprising: (a) isolating a tissue comprising a plurality of cells from a source, (b) subjecting said tissue to a medium comprising an agent capable of inducing cellular pathologies consistent with the pathologies in a patient suffering from said neuronal degenerative disease, (c) allowing sufficient contact time between said tissue and said medium, (d) identifying at least one cellular pathology present in patients suffering from said neuronal degenerative disease, (e) assigning a measurement to said identified pathology thereby correlating the severity of the neuronal degenerative disease in said patient.

2. The method of claim 1, wherein said neuronal degenerative disease is selected from the group consisting of ALS, Parkinson's disease (PD), Alzheimer's disease (AD), epilepsy and Huntington's disease (HD).

3. The method of claim 1, wherein said source is a mammal selected from the group consisting of rats, rabbits, guinea pigs and mice.

4. The method of claim 3, wherein said mammal is healthy, is at risk of developing said neuronal degenerative disorder, is suffering from said neuronal degenerative disease, or is transgenic.

5. The method of claim 1, wherein said agent is selected from the group consisting of inflammation associated reagents selected from the group consisting of histamine; TNF alpha;
lipopolysaccharide; aluminum chloride; serotonin; purine nucleotides selected from the group consisting of ATP, ADP, AMP, or combinations thereof; cytokines selected from the group consisting of interleukin 1a, growth factors selected from the group consisting of monocyte chemoattractant protein (MCP-1), activators of the phosphatidylinositol/Akt pathway selected from the group consisting of VEGF, oxidative stress associated reagents selected from the group consisting of generators of free radicals and nitric oxide, or extracts from natural compounds.

6. The method of claim 1, wherein said cellular pathologies is manifested by expression of a biomarker, expression of a cellular protein, or a change in tissue characteristics.

7. The method of claim 6, wherein said biomarker is abeta42, alpha 7 nicotinic acetyl choline receptors, IgG, autoantibodies, free radical species, cyclic nucleotides.

8. The method of claim 6, wherein said cellular proteins are cytoskeletal proteins comprising GFAP, MAP2 and vimentin, calcium binding proteins, S100B and visinin-like proteins, proteins that impact the cyclic GMP pathway, guanylate cyclases and their modulators.

9. The method of claim 6, wherein said changes in tissue characteristics include leaky blood vessels, change in thickness of a cellular layer, change in vascularization of a cellular layer, changes in dimensions including changes in length, thickness, area or any combinations thereof.

10. The method of claim 1, wherein sufficient time comprise a range of between 30 seconds to 1 month.

11. The method of claim 1, wherein the measuring of the cellular pathology is achieved through employing ultrasound, laser, staining, or patient surveys.

12. The method of claim 1, wherein the tissue is selected from the group consisting of brain tissue, retinal tissue, nasal tissue, skin tissue, and vascular tissue.

13. The method of claim 12, wherein the brain tissue is a brain slice.

14. The method of claim 13, wherein the brain slice is selected from the group consisting of a parenchymal slice, hippocampal slice, an entorhinal cortex slice, an entorhinohippocampal slice, a neocortex slice, a hypothlalamic slice, a cortex slice and combinations thereof.

15. A method for identifying a candidate compound for treatment of a patient at risk of developing a neuronal degenerative disease comprising: (a) isolating a tissue comprising a plurality of cells from a source, (b) subjecting said tissue to a medium comprising an agent capable of inducing cellular pathologies consistent with the pathologies in a patient suffering from said neuronal degenerative disease, (c) allowing sufficient contact time between said tissue and said medium, (d) identifying at least one cellular pathology present in patients suffering from said neuronal degenerative disease, (e) assigning a measurement to said identified pathology thereby correlating the severity of the neuronal degenerative disease in said patient (f) exposing said tissue to a test compound, (g) measuring the reversal, or the inhibition of the cellular pathology identified in step (e) .

16. The method of claim 15, further wherein the measurement observed in step (g) is further compared with a control brain tissue not contacted with said test agent.

17. The method of claim 15, wherein said neuronal degenerative disease is selected from the group consisting of ALS, Parkinson's disease (PD), Alzheimer's disease (AD), epilepsy and Huntington's disease (HD).

18. The method of claim 15, wherein said source is a mammal selected from the group consisting of a mammal selected from the group consisting of rats, rabbits, guinea pigs and mice.

19. The method of claim 18, wherein said mammal is healthy, is at risk of developing said neuronal degenerative disorder, is suffering from said neuronal degenerative disease, or is transgenic.

20. The method of claim 15, wherein said agent is selected from the group consisting of inflammation associated reagents selected from the group consisting of histamine; TNF alpha; lipopolysaccharide; aluminum chloride; serotonin; purine nucleotides selected from the group consisting of ATP, ADP, AMP, or combinations thereof; cytokines selected from the group consisting of interleukin 1a, growth factors selected from the group consisting of monocyte chemoattractant protein (MCP-1), activators of the phosphatidylinositol/Akt pathway selected from the group consisting of VEGF, oxidative stress associated reagents selected from the group consisting of generators of free radicals and nitric oxide, or extracts from natural compounds.

21. The method of claim 15, wherein said cellular pathologies is manifested by expression of a biomarker, expression of a cellular protein, or a change in tissue characteristics.

22. The method of claim 21, wherein said biomarker is GFAP, vimentin, abeta42, alpha 7 nicotinic acetyl choline receptors, IgG, autoantibodies, free radical species or combinations thereof.

23. The method of claim 21, wherein said cellular proteins are cytoskeletal proteins such as GFAP, MAP2 and vimentin, calcium binding proteins such as S100B and visinin-like proteins,proteins that impact the cyclic GMP pathway such as membrane guanylate cyclases and their modulators.

24. The method of claim 21, wherein said changes in tissue characteristics include leaky blood vessels, change in thickness of a cellular layer, change in vascularization of a cellular layer, changes in dimensions including changes in length, thickness, area and their appearance, organization, distribution or degeneration.

25. The method of claim 15, wherein sufficient time comprise a range of between 30 seconds to 1 month.

26. The method of claim 15, wherein the measuring of the cellular pathology is achieved through employing ultrasound, laser, or cellular staining.

27. A slice culture system for modeling a pathological condition comprising a plurality of tissue slices obtained from a suitable source in a receptacle, wherein said tissue slices are pre-treated with an effective amount of histamine sufficient to mimic the behavior of a tissue, and modify the expression of a biomarker expressed in a patient suffering from a pathological condition.

28. The slice culture system of claim 27, wherein the tissue slices are organotypic brain slices.
